(19) United States
(12) Patent Application Publication
(10) Pub. No.: US 2006/0052342 A1
(43) Pub. Date: Mar. 9, 2006

(54) Aldonic acid esters, methods for producing the same, and methods for producing pharmaceutical active ingredients coupled to polysaccharides or polysaccharide derivatives on free amino groups

(76) Inventor: Klaus Sommermeyer, Rosbach (DE)

Correspondence Address: HAMILTON, BROOK, SMITH & REYNOLDS, P.C., 530 VIRGINIA ROAD, P.O. BOX 9133, CONCORD, MA, 01742-9133, US

(21) Appl. No.: 10/537,176

(22) PCT Filed: Dec. 3, 2003
(86) PCT No.: PCT/EP03/13622

§ 371 Date: Sep. 2, 2005

(30) Foreign Application Priority Data
Dec. 4, 2002 (DE) 10256558.9

(51) Int. Cl. A61K 31/717; C08B 31/02

(52) U.S. Cl. 514/60; 536/110

(57) ABSTRACT

The invention relates to aldonic acid esters of starch fractions or starch fraction derivatives which are selectively oxidised on the reducing chain end to form aldonic acids, and to solids and solutions containing said aldonic acid esters. The invention also relates to methods for producing said aldonic acid esters, to methods for producing pharmaceutical active ingredients coupled to polysaccharides or polysaccharide derivatives on free amino functions, and to pharmaceutical active ingredients thus obtained.

The present invention relates to aldonic acid esters, solids and solutions which comprise these esters, and methods for the production thereof. The present invention further relates to methods for producing pharmaceutical active ingredients coupled to polysaccharides or polysaccharide derivatives on free amino groups, which are carried out using the aldonic acid esters, and to the pharmaceutically active ingredients obtainable by these methods.

The conjugation of pharmaceutical active ingredients in particular of proteins with polyethylene glycol derivatives (“PEGylation”) or polysaccharides such as dextrans or, in particular, hydroxyethyl starch (“HESylation”) has gained importance in recent years with the increase in pharmaceutical proteins from biotechnology research.

The biological half-life of such proteins is often too short but can be prolonged specifically by coupling to the abovementioned polymeric compounds such as PEG or HES. However, the coupling may also have a beneficial influence on the antigenic properties of proteins. In the case of other pharmaceutical active ingredients it is possible considerably to increase the solubility in water by the coupling.

DE 196 28 705 and DE 101 29 369 describe possible methods for carrying out the coupling of hydroxyethyl starch in anhydrous dimethyl sulfoxide (DMSO) via the corresponding aldonolactone of hydroxyethyl starch with free amino groups of hemoglobin and amphotericin B, respectively.

Since it is often not possible to use anhydrous, aprotic solvents specifically in the case of proteins, either for solubility reasons or else on the grounds of denaturation of the proteins, coupling methods with HES in an aqueous medium are also available. For example, coupling of hydroxyethyl starch which has been selectively oxidized at the reducing end of the chain to the aldonic acid is possible through the mediation of water-soluble carbodiimide EDC (1-ethyl-3-(3-dimethyl-aminopropyl)carbodiimide) (PCT/EP 02/02928). However, the use of carbodiimides is very often associated with disadvantages, because carbodiimides very frequently cause inter- or intramolecular crosslinking reactions of the proteins as side reactions.

In the case of compounds containing phosphate groups, such as nucleic acids, the coupling is often impossible because the phosphate groups may likewise react with EDC (S. S. Wong, Chemistry of Protein Conjugation and Cross-Linking, CRC-Press, Boca Raton, London, New York, Washington D.C., 1993, page 199).

In view of the discussed prior art, the object on which the invention was based was to provide compounds which specifically make it possible, avoiding the previously described disadvantages, to couple polysaccharides or their derivatives to active ingredients containing amino groups, especially to proteins, in purely aqueous systems or else in solvent mixtures with water.

It was additionally intended that the nature of such a compound be such that the attachment of an active ingredient by covalent bonding to a polysaccharide or a polysaccharide derivative is as quantitative as possible.

The invention was additionally based on the object of providing compounds which make it possible to link a polysaccharide or a derivative thereof to the active ingredient under conditions which are as mild as possible. Thus, in particular, the reaction was intended to change as little as possible in the structure, the activity and the tolerability of the active ingredient. For example, intra- and intermolecular crosslinking reactions were to be avoided. In addition, it was also intended to be able to link active ingredients which have phosphate groups.

It was therefore also an object of the present invention to indicate compounds which permit coupling as selectively as possible to the active ingredient. Thus, it was intended in particular to be able to adjust a specific stoichiometry of the conjugate, it being specifically intended to make it possible to prepare 1:1 conjugates through the use of these compounds.

Finally, the invention was based on the object of providing a method which is as simple and cost-effective as possible for preparing such compounds and coupling products of polysaccharides or polysaccharide derivatives with active ingredients.

These objects and others which, although not mentioned verbatim, can be inferred as self-evident from the contexts discussed herein, or are automatically evident therefrom, are achieved with the aldonic acid esters described in claim 1. Expedient modifications of these aldonic acid esters according to the invention, and stable aldonic acid esters which can be employed in methods for preparing conjugates, are protected by dependent claims 2-19 which refer back to claim 1.

In relation to a method for preparing the aldonic acid esters, claims 20-28 provide an achievement of the underlying object.

Claims 29-34 describe methods for preparing polysaccharide-active ingredient conjugates and the pharmaceutical active ingredients obtainable by these methods.

The provision of aldonic acid esters which are derived from polysaccharides or polysaccharide derivatives which are selectively oxidized at the reducing end of the chain to aldonic acids allows compounds which achieve the aforementioned objects to be provided. Such esters can be regarded as activated acids. They react in an aqueous medium with nucleophilic NH2 groups to give (more stable) amides.

In addition, the following advantages inter alia are achieved by the present invention:

The aldonic acid esters of the invention make it possible easily to attach an active ingredient by covalent bonding to a polysaccharide or a polysaccharide derivative takes place.

The aldonic acid esters of the present invention can be reacted with an active ingredient under mild conditions. In this case, in particular the structure, the activity and the tolerability of the active ingredient is changed to only a small extent by the reaction. It is possible in this way inter alia to avoid in particular intra- and intermolecular crosslinking reactions. A further possibility is to couple pharmaceutical active ingredients which have phosphate groups without these groups being changed.

The aldonic acid esters of the invention permit very selective coupling to the active ingredient. It is additionally possible for example to adjust a specific stoichiometry of the desired conjugate, the use of these compounds making it possible specifically to prepare 1:1 conjugates.

The present invention additionally provides simple and cost-effective methods for preparing activated aldonic acid esters and coupling products of polysaccharides or polysaccharide derivatives with active ingredients.

The aldonic acid esters of the present invention are derived from polysaccharides or polysaccharide derivatives which can be selectively oxidized at the reducing end of the chain. Polysaccharides of this type, and derivatives obtainable therefrom, are widely known in the art and can be obtained commercially. Polysaccharides are macromolecular carbohydrates whose molecules have a large number (min. >10, but usually considerably more) monosaccharide molecules (glycose) glycosidically linked together. The weight average molecular weight of preferred polysaccharides is preferably in the range from 1500 to 1 000 000 Dalton, particularly preferably 2000 to 300 000 Dalton and very particularly preferably in the range from 2000 to 50 000 Dalton. The molecular weight Mw determined by usual methods. These include for example aqueous GPC, HPLC, light scattering and the like.

It is possible inter alia to change the residence time in the body via the molecular weight of the polysaccharide residue.

The preferred polysaccharides include starch and the starch fractions obtainable by hydrolysis, which can be regarded as starch degradation products. Starch is normally divided into amylose and amylopectin which differ in the degree of branching. Amylopectin is particularly preferred according to the invention.

Amylopectins mean in this connection in the first place very generally branched starches or starch products with a-(1-4) and a-(1-6) linkages between the glucose molecules. The branchings of the chain take place via the a-(1-6) linkages. These are present irregularly approximately every 15-30 glucose segments in naturally occurring amylopectins. The molecular weight of natural amylopectin is very high in the range from 107 to 2′ 108 Dalton. It is assumed that amylopectin also forms helices within certain limits.

A degree of branching can be defined for amylopectins. The measure of the branching is the ratio of the number of molecules of anhydroglucose which have branch points (a-(1-6) linkages) to the total number of molecules of anhydroglucose in the amylopectin, this ratio being expressed in mol %. Naturally occurring amylopectin has degrees of branching of about 4 mol %. Amylopectins preferably employed for preparing the aldonic acid esters have an average branching in the range from 5 to 10 mol %.

It is additionally possible to employ hyperbranched amylopectins which have a degree of branching which significantly exceeds the degree of branching known for amylopectins in nature. In this connection, the degree of branching is in every case an average (average degree of branching), because amylopectins are polydisperse substances.

Such hyperbranched amylopectins have significantly higher degrees of branching expressed as mol % of the branching anhydroglucoses by comparison with unmodified amylopectin or hydroxyethyl starch and are accordingly more similar in their structure to glycogen.

The average degree of branching of the hyperbranched amylopections is normally in the range between >10 and 25 mol %. This means that these amylopectins have on average an a-(1-6) linkage, and thus a branch point, about every 10 to 4 glucose units. A preferred amylopectin type which can be employed in the medical sector is characterized by a degree of branching of between 11 and 16 mol %.

Further preferred hyperbranched amylopectins have a degree of branching in the range between 13 and 16 mol %.

The amylopectins which can be employed in the invention preferably have a value for the weight average molecular weight Mw in the range from 2000 to 800 000 Dalton, in particular 2000 to 300 000 and particularly preferably 2000 to 50 000 Dalton.

The starches described above can be obtained commercially. Isolation thereof is moreover known from the literature. Thus, starch can be isolated in particular from potatoes, tapioca, manioc, rice, wheat or corn. The starches obtained from these plants are often initially subjected to a hydrolytic degradation reaction. During this, the molecular weight is reduced from about 20 000 000 Dalton to several million Dalton, and a further degradation of the molecular weight to the aforementioned values is likewise known. It is possible and particularly preferred inter alia for waxy corn starch degradation fractions to be employed for preparing the aldonic acid esters of the invention.

The hyperbranched starch fractions described above are described inter alia in the German patent application 102 17 994.

It is additionally possible to employ derivatives of polysaccharides for preparing the aldonic acid esters of the invention. These include in particular hydroxyalkyl starches, for example hydroxyethyl starch and hydroxypropyl starch, which can be obtained by hydroxyalkylation from the starches described above, in particular from amylopectin. Of these, hydroxyethyl starch (HES) is preferred.

The HES preferably employed according to the invention is the hydroxyethylated derivative of amylopectin which is the glucose polymer which constitutes more than 95% of waxy corn starch. Amylopectin consists of glucose units which are present in a-1,4-glycosidic linkages and have a-1,6-glycosidic branches.

HES has advantageous rheological properties and is currently used clinically as volume replacement agent and for hemodilution therapy (Sommermeyer et al., Krankenhauspharmazie, Vol. 8 (8, 1987) pages 271-278 and Weidler et al., Arzneimittelforschung/Drug Res., 41, (1991) pages 494-498).

HES is characterized essentially via the weight average molecular weight Mw, the number average molecular weight Mn, the molecular weight distribution and the substitution level. Substitution with hydroxyethyl groups in ether linkage is in this case possible at carbon atoms 2, 3 and 6 of the anhydroglucose units. The substitution level can in this connection be described as DS (“degree of substitution”) which is based on the substituted glucose molecules as a proportion of all the glucose units, or as MS (“molar substitution”) which refers to the average number of hydroxyethyl groups per glucose unit.

The substitution level MS (molar substitution) is defined as the average number of hydroxyethyl groups per anhydroglucose unit. It is measured from the total number of hydroxyethyl groups in a sample, for example by the method of Morgan, by ether cleavage and subsequent quantitative determination of ethyl iodide and ethylene which are formed thereby.

By contrast, the substitution level DS (degree of substitution) is defined as the substituted anhydroglucose units as a proportion of all anhydroglucose units. It can be determined from the measured amount of unsubstituted glucose after hydrolysis of a sample. It is evident from these definitions that MS>DS. In the case where only monosubstitution is present, that is each substituted anhydroglucose unit has only one hydroxyethyl group, MS=DS.

A hydroxyethyl starch residue preferably has a substitution level MS of from 0.1 to 0.8. The hydroxyethyl starch residue particularly preferably has a substitution level MS of from 0.4 to 0.7.

The reactivity of the individual hydroxy groups in the unsubstituted anhydroglucose unit for hydroxyethylation differs depending on the reaction conditions. It is possible thereby within certain limits to influence the substitution pattern, that is the individual differently substituted anhydroglucoses which are randomly distributed over the individual polymer molecules. It is advantageous for the C2 position and the C6 position to be predominantly hydroxyethylated, with the C6 being substituted more often because of its easier accessibility.

It is preferred to use for the purposes of this invention hydroxyethyl starches (HES) which are predominantly substituted in the C2 position and which are substituted as homogeneously as possible. The preparation of such HES is described in EP 0 402 724 B2. They are completely degradable within a physiologically reasonable time and, on the other hand, nevertheless display controllable elimination behavior. The predominant C2 substitution makes it relatively difficult for a-amylase to degrade the hydroxyethyl starch. It is advantageous where possible for no consecutively substituted anhydroglucose units to occur within the polymer molecule, in order to ensure complete degradability. In addition, despite the low substitution, such hydroxyethyl starches have sufficiently high solubility in aqueous medium, so that the solutions are also stable over prolonged periods and no agglomerates or gels form.

Based on the hydroxyethyl groups of the anhydroglucose units, a hydroxyethyl starch residue preferably has a C2:C6 substitution ratio in the range from 2 to 15. The C2:C6 substitution ratio is particularly preferably from 3 to 11.

Selective oxidation of the aldehyde group of the polysaccharides or polysaccharide derivatives described above to the aldonic acid is known per se. This can be effected by mild oxidizing agents, for example iodine/potassium hydroxide in accordance with DE 196 28 705 A1, or by enzymes.

The free aldonic acid can be employed for the reaction. It is also possible additionally to employ salts. These include in particular the alkali metal salts such as, for example, the sodium and/or the potassium salt of the aldonic acids.

Alcohols are employed to prepare the aldonic acid esters of the invention. The term alcohol includes compounds which have HO groups. These HO groups may be bonded inter alia to a nitrogen atom or to a phenyl radical.

Acidic alcohols which are known in the art are preferably employed. These include inter alia N-hydroxyimides, for example N-hydroxysuccinimide and sulfo-N-hydroxysuccinimide, substituted phenols and hydroxyazoles, for example hydroxybenzotriazole, with particular preference for N-hydroxysuccinimides and sulfo-N-hydroxysuccinimide.

Further suitable acidic alcohols for preparing the aldonic acid esters of the invention are detailed in the literature. (V. H. L. Lee, Ed., Peptide and Protein Drug Delivery, Marcel Dekker, 1991, p. 65).

In a particular aspect of the present invention, alcohols whose HO group has a pka in the range from 6 to 12, preferably in the range from 7 to 11, are employed. This value refers to the acid dissociation constant determined at 25° C., this value being quoted many times in the literature.

The molecular weight of the alcohol is preferably in the range from 80 to 500 g/mol, in particular 100 to 200 g/mol.

The alcohol can be added as free to a reaction mixture. It is also possible to use for the reaction compounds which release alcohol on addition of water, where appropriate with acid catalysis.

In a particular aspect of the present invention, carbonic diesters are employed for the reaction with the aldonic acid or an aldonic acid salt. These compounds enable the reaction to be particularly rapid and mild, with formation only of carbonic acid or carbonates, alcohols and the desired aldonic acid ester.

Preferred carbonic diesters are, inter alia, N′N-succinimidyl carbonate and sulfo-N′N-succinimidyl carbonate.

These carbonic diesters can be employed in relatively small amounts. Thus, the carbonic diester can be employed in a 1- to 3-molar excess, preferably 1 to 1.5 molar excess, based on the aldonic acid and/or the aldonic acid salt. The reaction time on use of carbonic diesters is relatively short. Thus, the reaction may in many cases be complete after 2 hours, preferably after 1 hour.

The reaction to give the aldonic acid ester preferably takes place in an anhydrous aprotic solvent. The water content should preferably not exceed 0.5% by weight, particularly preferably not exceed 0.1% by weight. Suitable solvents are, inter alia, dimethyl sulfoxide (DMSO), N-methylpyrrolidone, dimethylacetamide (DMA) and/or dimethylformamide (DMF).

The esterification reaction is known per se, it being possible to employ any method. The reaction to give the aldonic acid ester can take place inter alia with use of activating compounds. Such a procedure is advisable on use of the free alcohol. The activating compounds include in particular carbodiimide such as, for example, dicyclohexylcarbodiimide (DCC) and 1-ethyl-3-(3-dimethylaminopropyl)carbodiimide (EDC).

On use of the free alcohol, the latter can be employed in a molar excess. In a particular aspect of the present invention, the alcohol component is preferably employed in a 5 to 50-fold molar excess, particularly preferably 8 to 20-fold excess based on the aldonic acid and/or the aldonic acid derivative.

The reaction to give the aldonic acid ester proceeds under mild conditions. Thus, the reactions described above can be carried out at temperatures preferably in the range from 0° C. to 40° C., particularly preferably 10° C. to 30 C.

In a particular aspect of the present invention, the reaction takes place with a low base activity. The low base activity can be measured by adding the reaction mixture to a 10-fold excess of water. In this case, the water has a pH of 7.0 at 25° C. before the addition, the water essentially comprising no buffer. The base activity of the reaction mixture is obtained by measuring the pH at 25° C. after addition of the reaction mixture. The pH of this mixture after addition is preferably no higher than 9.0, particularly preferably no higher than 8.0 and particularly preferably no higher than 7.5.

The reaction with HES-aldonic acids, e.g. with N-hydroxysuccinimide, proceeds in dry DMA, excluding water, with EDC in a smooth reaction at room temperature to give the HES-acid N-hydroxysuccinimide ester. It is particularly surprising in this connection that no side reaction of the HES molecule occurs through reaction of the OH groups of the anhydroglucoses, which are present in extreme excess, with EDC, and the rearrangement reaction of the initially formed O-acyl isourea from EDC and the aldonic acid to the corresponding N-acyl urea is suppressed.

The solutions obtained by the reaction described above can be employed in coupling reactions without isolation of the aldonic acid esters. Since the volume of the preactivated aldonic acid in the aprotic solvent is usually small compared with the target protein dissolved in the buffer volume, the amounts of aprotic solvent in most cases have no interfering effect. Preferred solutions include at least 10% by weight aldonic acid ester, preferably at least 30% by weight aldonic acid ester and particularly preferably at least 50% by weight aldonic acid ester.

The aldonic acid esters can be precipitated from the solution in the aprotic solvent, for example DMA, by known precipitants such as, for example, dry ethanol, isopropanol or acetone and be purified by repetition of the procedure more than once. Preferred solids include at least 10% by weight aldonic acid ester, preferably at least 30% by weight aldonic acid ester and particularly preferably 50% by weight aldonic acid ester.

Such aldonic acid esters can then be isolated as substance for coupling, for example for HESylation. During this, no side reactions as described above with EDC-activated acid occur.

For the coupling it is additionally possible to add a solution of the activated aldonic acid to an aqueous solution of the pharmaceutical active ingredient, which is preferably buffered, at a suitable pH. The pharmaceutical active ingredients include at least one amino group which can be reacted to give the aldonamide. The preferred active ingredients include proteins and peptides.

The pH of the reaction depends on the properties of the active ingredient. The pH is preferably, if possible, in the range from 7 to 9, particularly preferably 7.5 to 8.5.

The coupling generally takes place at temperatures in the range from 0° C. to 40° C., preferably 10° C. to 30° C., without this intending to introduce a restriction. The reaction time can easily be ascertained by suitable methods. The reaction time is generally in the range from 1 hour to 100 hours, preferably 20 hours to 48 hours.

The aldonic acid ester can be employed in an excess in relation to the pharmaceutical active ingredient. The aldonic acid ester is preferably employed in a 1 to 5-fold molar excess, particularly preferably 1.5 to 2-fold excess, based on the pharmaceutical active ingredient.

Essentially the only byproduct in the abovementioned reaction is the alcohol, for example N-hydroxysuccinimide, which can easily be separated from the coupling product, e.g. by ultrafiltration. A side reaction which may occur is hydrolysis with water to the free acid and to the free alcohol. It is therefore particularly surprising that the aldonic acid esters of the invention to a large extent enters into a coupling reaction with a pharmaceutical active ingredient. This is evident from the examples, in particular through the chromatograms depicted in the figures.

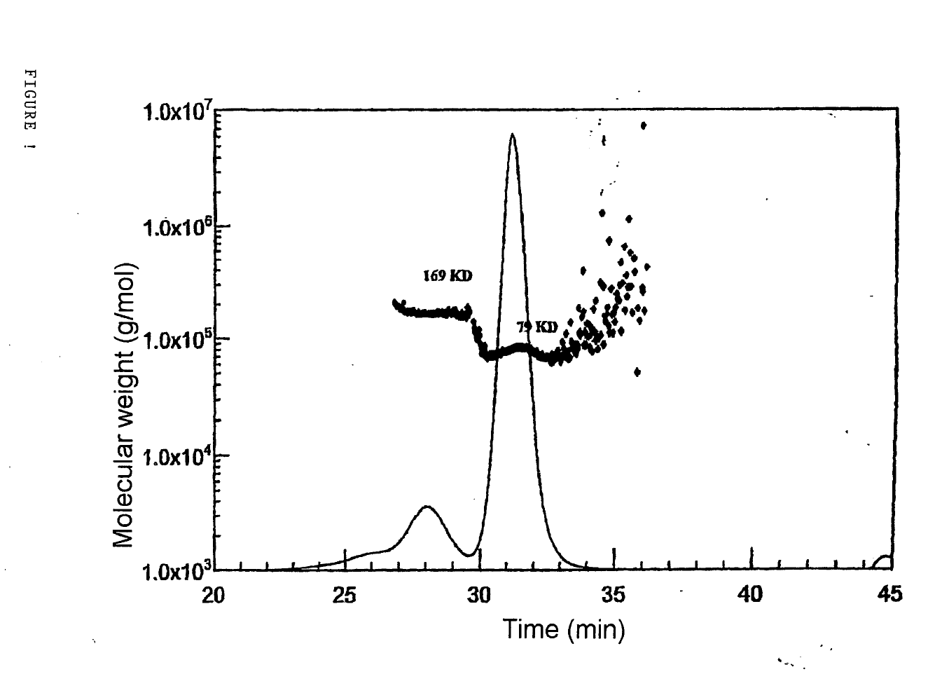

FIG. 1 MALLS-GPC chromatogram of unreacted bovine albumin (BSA). Monomeric and dimeric albumin are clearly separated.

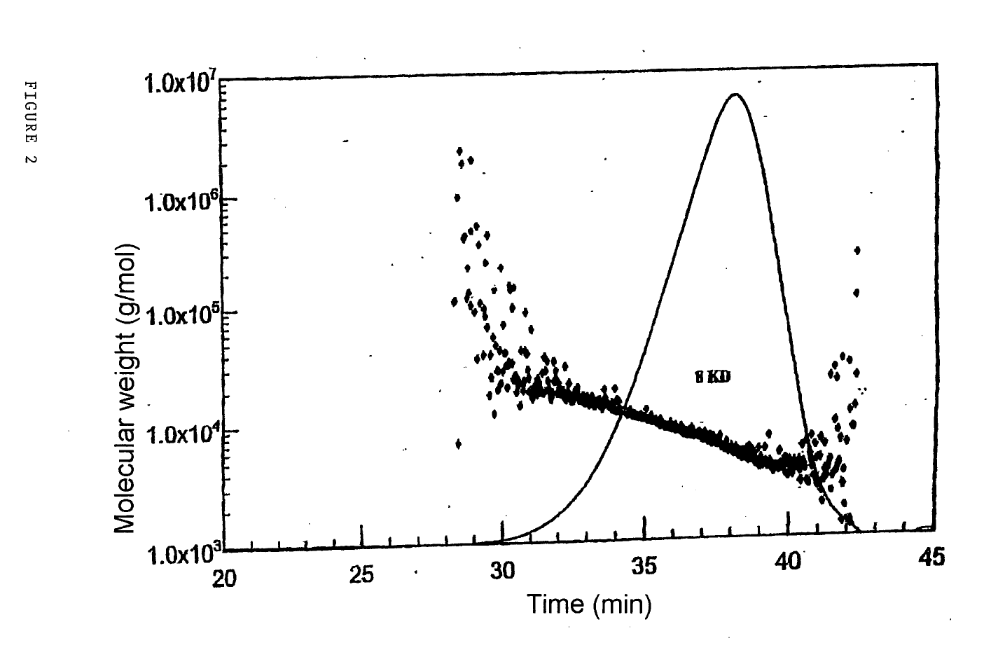

FIG. 2 MALLS-GPC chromatogram of unreacted HES-10/0.4-succinimidyl ester.

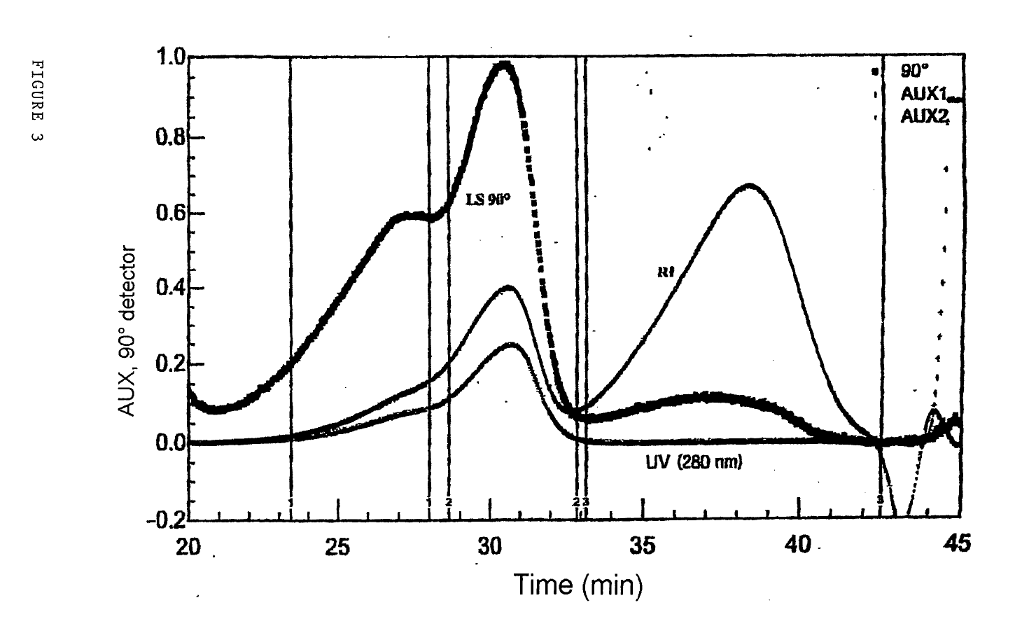

FIG. 3 MALLS-GPC chromatogram of the product of the reaction of HES-10/0.4-succinimidyl ester and BSA. The signals shown are those of the 3-fold detection of refractive index (RI), UV detector and the light scattering signal at 90°.

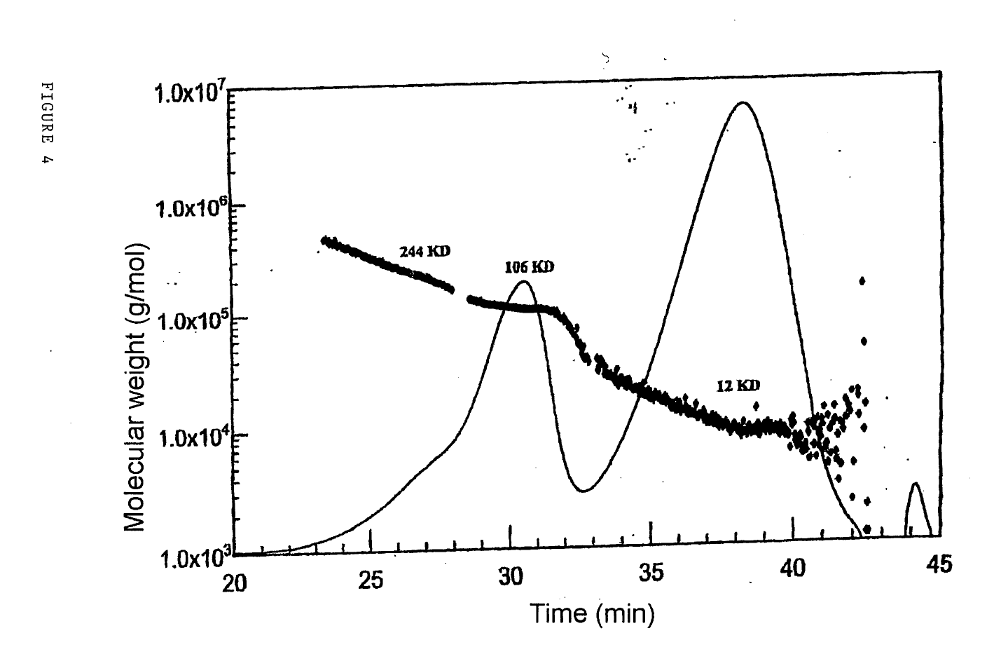

FIG. 4 MALLS-GPC chromatogram of the product of the reaction of HES-10/0.4-succinimidyl ester and BSA, representing molecular mass against time.

The invention is explained in more detail below by examples and comparative examples without intending to restrict the invention to these examples.

### EXAMPLES AND PREPARATION METHODS

### Example 1

### Preparation of HES 10/0.4-Acid Esters with N-hydroxysuccinimide

5 g of dry hydroxyethyl starch with an average molecular weight Mw=10 000 Dalton and a substitution level MS=0.4, which has been selectively oxidized at the terminal reducing end of the chain in accordance with DE 196 28 705, are dissolved in 30 ml of dry dimethylacetamide at 40° C. and, after cooling of the solution, 10 times the molar amounts of N-hydroxysuccinimide are added with exclusion of moisture. The amount of EDC equimolar to the HES acid is then added in portions, and the reaction mixture is allowed to react to completion 24 hours after the addition. The reaction product is subsequently precipitated with dry acetone and purified by repeated reprecipitation.

### Example 2

### Preparation of Hes 10/0.4-Acid Coupled Myoglobin

15 mg of myoglobin are dissolved in 20 ml of distilled water, and the pH is adjusted to 7.5 with sodium hydroxide solution. 1.5 g of HES 10/0.4-acid N-hydroxysuccinimide, prepared as in Example 1, are added in portions to the solution over the course of 1 hour, and the pH is kept constant at 7.5 by adding sodium hydroxide solution.

The mixture is left to stir overnight.

The formation of hesylated myoglobin is determined by gel permeation chromatography with a yield of 70% based on the myoglobin employed.

### Example 3

### Preparation of HES 10/0.4-acid ester with N′N-disuccinimidyl carbonate

0.02 mmol (equivalent to 0.14 g) of dried HES 10/0.4-acid is dissolved in 2 ml of dried dimethylformamide with exclusion of moisture. 0.02 mmol of N′N-disuccinimidyl carbonate is added to the solution, and reaction is allowed to go to completion at room temperature with stirring for 1 hour.

### Example 4

### Preparation of the Coupling Product of HES 10/0.4-acid with Bovine Serum Albumin

50 mg of bovine serum albumin (BSA equivalent to 0.7 mmol) are dissolved in 6 ml of a 0.3 molar bicarbonate solution of pH 8.4. The mixture from Example 3 is added to the solution, and the reaction is allowed to go to completion by stirring at room temperature for 2 hours.

Demonstration that the reaction has succeeded takes place by low pressure HPGPC with multiple detection (UV 280 nm, MALLS light scattering detector (MALLS=multiangle laser light scattering), RI detector).

FIGS. 1 to 4 show for comparison the chromatograms of the unreacted HES 10/0.4-succinimidyl esters, the starting material BSA and the reaction mixture.

Success of the reaction is evident from a significant decrease in the BSA peak and the appearance of a higher molecular weight peak which is detected at 280 nm.

### Example 5

### Preparation of HES 50/0.7-acid ester with N′N-disuccinimidyl carbonate

0.02 mmol (0.5 g) of dried HES 50/0.7-acid is dissolved in 2 ml of dried dimethylformamide with exclusion of moisture. 0.02 mmol of N′N-disuccinimidyl carbonate is added to the solution, and reaction is allowed to go to completion at room temperature with stirring for 1 hour.

### Example 6

### Preparation of HES 50/0.7 Coupling Product with BSA

50 mg of bovine serum albumin BSA (0.7 mmol) are dissolved in 6 ml of a 0.3 molar bicarbonate solution of pH 8.4. The solution of the activated HES 50/0.7-acid from Example 5 is added to the solution, and reaction is allowed to go to completion by stirring at room temperature for 2 hours.

Analytical monitoring of the reaction mixture takes place by low pressure HPGPC with triple detection as described in Example 4.

Success of the reaction is evident from a decrease in the signal at 280 nm for unreacted BSA and the corresponding appearance of the signal shifted to higher molecular weights for the coupling product. The shift is larger than in Example 4 in accordance with the higher molecular weight of the HES acid.

1-34. (canceled)

35. An aldonic acid ester of polysaccharides or polysaccharide derivatives which are selectively oxidized at the reducing end of the chain to aldonic acids.

36. The aldonic acid ester as claimed in claim 35, wherein the polysaccharides or polysaccharide derivatives are starch fractions or starch fraction derivatives.

37. The aldonic acid ester as claimed in claim 36, wherein the starch fractions are amylopectin degradation fractions.

38. The aldonic acid ester as claimed in claim 37, wherein the amylopectin degradation fractions are obtained by acid degradation and/or degradation by α-amylase of waxy corn starch.

39. The aldonic acid ester as claimed in claim 38, wherein the starch fractions have an average molecular weight MW of 2000-50 000 Dalton and an average branching of 5-10 mol % α-1,6-glycosidic linkages.

40. The aldonic acid ester as claimed in claim 38, wherein the starch fractions have an average molecular weight MW of 2000-50 000 Dalton and an average branching in the range of greater than 10 to 25 mol % α-1,6-glycosidic linkages.

41. The aldonic acid ester as claimed in claim 36, wherein the starch fraction derivatives are hydroxyethyl derivatives of waxy corn starch degradation fractions.

42. The aldonic ester as claimed in claim 41, wherein the average molecular weight MW of the hydroxyethyl starch fractions is in the range of 2-300 000 Dalton, and the substitution level MS is between 0.1 and 0.8, and the C2/C6 ratio of the substituents on carbon atoms C2 and C6 of the anhydroglucoses is between 2 and 15.

43. The aldonic acid ester as claimed in claim 35 wherein the alcohol from which the alcohol component of the aldonic acid ester is derived has a molecular weight in the range from 80 to 500 g/mol.

44. The aldonic acid ester as claimed in claim 35, wherein the alcohol from which the alcohol component of the aldonic acid ester is derived has a pKa in the range from 6 to 12.

45. The aldonic ester as claimed in claim 35, wherein the alcohol from which the alcohol component of the aldonic acid ester is derived, of the aldonic acid ester, includes an HO—N group or a phenol group.

46. The aldonic acid ester as claimed in claim 35, wherein the alcohol from which the alcohol component of the aldonic acid ester is derived is selected from N-hydroxysuccinimide, sulfo-N-hydroxysuccinimide, substituted phenols and hydroxybenzotriazole.

47. The aldonic acid ester as claimed in claim 46, wherein the alcohol from which the alcohol component of the aldonic acid ester is derived is N-hydroxysuccinimide and sulfo-N-hydroxysuccinimide.

48. A solid comprising at least one aldonic acid ester as claimed in claim 35.

49. A solution comprising at least one aldonic acid ester as claimed in claim 35.

50. The solution as claimed in claim 49, wherein the solution comprises at least one organic solvent.

51. The solution as claimed in claim 50, wherein the solution comprises not more than 0.5% by weight water.

52. The solution as claimed in claim 49, wherein the solution comprises at least one aprotic solvent.

53. The solution as claimed in claim 52, wherein the solvent is dimethyl sulfoxide (DMSO), N-methylpyrrolidone, dimethylacetamide (DMA) and/or or dimethylformamide (DMF).

54. A method for preparing aldonic acid ester as claimed in claim 35, wherein at least one aldonic acid and/or one aldonic acid derivative is reacted with at least one alcohol component in aprotic solvent.

55. The method as claimed in claim 54, wherein the alcohol component is employed in 5 to 50-fold molar excess based on that aldonic acid and/or the aldonic acid derivative.

56. The method as claimed in claim 54, wherein the reaction takes place with the use of at least one activating reagent.

57. The method as claimed in claim 56, wherein the activating reagent comprises at least one carbodiimide.

58. The method as claimed in claim 56, wherein the activating reagent is employed in 1- to 3-molar excess based on the aldonic acid and/or the aldonic acid derivative.

59. The method as claimed in claim 54, wherein a compound which liberates an alcohol component for reaction with the aldonic acid or the aldonic acid derivative is employed.

60. The method as claimed in claim 59, wherein a carbonic diester is employed.

61. The method as claimed in claim 54, wherein the reaction takes place at a temperature in the range from 0 to 40° C.

62. The method as claimed in claim 54, wherein the reaction takes place at a low base activity.

63. A method for preparing pharmaceutical active ingredients coupled to polysaccharides or polysaccharide derivatives on free amino functions, wherein at least one aldonic acid ester as claimed in claim 35 is reacted with a pharmaceutical active ingredient which has at least one amino group.

64. The method as claimed in claim 63, wherein the reaction takes placed in aqueous medium.

65. The method as claimed in claim 64, wherein the pH of the aqueous medium is in the range from 7 to 9.

66. The method as claimed in claim 63, wherein the reaction takes place at a temperature in the range from 0° C. to 40° C.

67. The method as claimed in claim 63, wherein the pharmaceutical active ingredient is a polypeptide or a protein.

68. A pharmaceutical active ingredient which is coupled to polysaccharides or polysaccharide derivatives and is obtained by the method as claimed in claim 63, wherein the pharmaceutical active ingredient is denatured in anhydrous medium and enters into unwanted side reactions with carbodiimides, such as inter- and intramolecular crosslinking or reaction with phosphate groups of the pharmaceutical active ingredient.
